SEQUENCE LISTING (54 sequences)

SEQ ID NO: 1 - DNA, 20 bp, Artificial Sequence
  Description of Artificial Sequence Synthetic oligonucleotide
gttcactgccgtataggcag

SEQ ID NO: 2 - DNA, 28 bp, Artificial Sequence
  Description of Artificial Sequence Synthetic oligonucleotide
gttcactgccgtataggcagctaagaaa

SEQ ID NO: 3 - protein, 4 aa, Artificial Sequence
  Description of Artificial Sequence Synthetic peptide
GGGS

SEQ ID NO: 4 - RNA, 342 nt, Artificial Sequence
  Description of Artificial Sequence Synthetic polynucleotide
  modified_base (1)..(20): a, c, u or g and this region may encompass 17-20 nucleotides wherein some positions may be absent
  modified_base (43)..(342): a, c, u or g and this region may encompass 0-300 nucleotides wherein some positions may be absent
nnnnnnnnnnnnnnnnnnnnguuuuagagcuaugcuguuuugnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 5 - RNA, 32 nt, Artificial Sequence
  Description of Artificial Sequence Synthetic oligonucleotide
  modified_base (1)..(20): a, c, u or g and this region may encompass 17-20 nucleotides wherein some positions may be absent
nnnnnnnnnnnnnnnnnnnnguuuuagagcua

SEQ ID NO: 6 - RNA, 42 nt, Artificial Sequence
  Description of Artificial Sequence Synthetic oligonucleotide
  modified_base (1)..(20): a, c, u or g and this region may encompass 17-20 nucleotides wherein some positions may be absent
nnnnnnnnnnnnnnnnnnnnguuuuagagcuaugcuguuuug

SEQ ID NO: 7 - RNA, 36 nt, Artificial Sequence
  Description of Artificial Sequence Synthetic oligonucleotide
  modified_base (1)..(20): a, c, u or g and this region may encompass 17-20 nucleotides wherein some positions may be absent
nnnnnnnnnnnnnnnnnnnnguuuuagagcuaugcu

SEQ ID NO: 8 - RNA, 362 nt, Artificial Sequence
  Description of Artificial Sequence Synthetic polynucleotide
  modified_base (1)..(20): a, c, u or g and this region may encompass 17-20 nucleotides wherein some positions may be absent
  modified_base (63)..(362): a, c, u or g and this region may encompass 0-300 nucleotides wherein some positions may be absent
nnnnnnnnnnnnnnnnnnnnguuuuagagcuagaaauagcaaguuaaaauaaggcuaguccgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 9 - RNA, 375 nt, Artificial Sequence
  Description of Artificial Sequence Synthetic polynucleotide
  modified_base (1)..(20): a, c, u or g and this region may encompass 17-20 nucleotides wherein some positions may be absent
  modified_base (76)..(375): a, c, u or g and this region may encompass 0-300 nucleotides wherein some positions may be absent
nnnnnnnnnnnnnnnnnnnnguuuuagagcuaugcugaaaagcauagcaaguuaaaauaaggcuaguccguuaucnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 10 - RNA, 387 nt, Artificial Sequence
  Description of Artificial Sequence Synthetic polynucleotide
  modified_base (1)..(20): a, c, u or g and this region may encompass 17-20 nucleotides wherein some positions may be absent
  modified_base (88)..(387): a, c, u or g and this region may encompass 0-300 nucleotides wherein some positions may be absent
nnnnnnnnnnnnnnnnnnnnguuuuagagcuaugcuguuuuggaaacaaaacagcauagcaaguuaaaauaaggcuaguccguuaucnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 11 - RNA, 396 nt, Artificial Sequence
  Description of Artificial Sequence Synthetic polynucleotide
  modified_base (1)..(20): a, c, u or g and this region may encompass 17-20 nucleotides wherein some positions may be absent
  modified_base (97)..(396): a, c, u or g and this region may encompass 0-300 nucleotides wherein some positions may be absent
nnnnnnnnnnnnnnnnnnnnguuuuagagcuagaaauagcaaguuaaaauaaggcuaguccguuaucaacuugaaaaaguggcaccgagucggugcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 12 - RNA, 96 nt, Artificial Sequence
  Description of Artificial Sequence Synthetic oligonucleotide
  modified_base (1)..(20): a, c, u or g and this region may encompass 17-20 nucleotides wherein some positions may be absent
nnnnnnnnnnnnnnnnnnnnguuuaagagcuagaaauagcaaguuuaaauaaggcuaguccguuaucaacuugaaaaaguggcaccgagucggugc

SEQ ID NO: 13 - RNA, 106 nt, Artificial Sequence
  Description of Artificial Sequence Synthetic polynucleotide
  modified_base (1)..(20): a, c, u or g and this region may encompass 17-20 nucleotides wherein some positions may be absent
nnnnnnnnnnnnnnnnnnnnguuuuagagcuaugcuggaaacagcauagcaaguuuaaauaaggcuaguccguuaucaacuugaaaaaguggcaccgagucggugc

SEQ ID NO: 14 - RNA, 106 nt, Artificial Sequence
  Description of Artificial Sequence Synthetic polynucleotide
  modified_base (1)..(20): a, c, u or g and this region may encompass 17-20 nucleotides wherein some positions may be absent
nnnnnnnnnnnnnnnnnnnnguuuaagagcuaugcuggaaacagcauagcaaguuuaaauaaggcuaguccguuaucaacuugaaaaaguggcaccgagucggugc

SEQ ID NO: 15 - DNA, 36 bp, Artificial Sequence
  Description of Artificial Sequence Synthetic oligonucleotide
  Description of Combined DNA/RNA Molecule Synthetic oligonucleotide
  modified_base (1)..(20): a, c, u or g and this region may encompass 17-20 nucleotides wherein some positions may be absent
nnnnnnnnnnnnnnnnnnnngttttagagctagaaa

SEQ ID NO: 16 - DNA, 60 bp, Artificial Sequence
  Description of Artificial Sequence Synthetic oligonucleotide
tagcaagttaaaataaggctagtccgttatcaacttgaaaaagtggcaccgagtcggtgc

SEQ ID NO: 17 - protein, 5 aa, Artificial Sequence
  Description of Artificial Sequence Synthetic peptide
GGGGS

SEQ ID NO: 18 - protein, 13 aa, Artificial Sequence
  Description of Artificial Sequence Synthetic peptide
DAYPYDVPDYASL

SEQ ID NO: 19 - protein, 17 aa, Artificial Sequence
  Description of Artificial Sequence Synthetic peptide
PKKKRKVEDPKKKRKVD

SEQ ID NO: 20 - protein, 1401 aa, Artificial Sequence
  Description of Artificial Sequence Synthetic polypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVD

SEQ ID NO: 21 - protein, 1368 aa, Artificial Sequence
  Description of Artificial Sequence Synthetic polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 22 - protein, 4 aa, Artificial Sequence
  Description of Artificial Sequence Synthetic peptide
GGSG

SEQ ID NO: 23 - protein, 12 aa, Artificial Sequence
  Description of Artificial Sequence Synthetic peptide
SGSETPGTSESA

SEQ ID NO: 24 - protein, 16 aa, Artificial Sequence
  Description of Artificial Sequence Synthetic peptide
SGSETPGTSESATPES

SEQ ID NO: 25 - protein, 21 aa, Artificial Sequence
  Description of Artificial Sequence Synthetic peptide
SGSETPGTSESATPEGGSGGS

SEQ ID NO: 26 - DNA, 24 bp, Artificial Sequence
  Description of Artificial Sequence Synthetic oligonucleotide
gccgaggtgaagttcgagggcgac

SEQ ID NO: 27 - DNA, 23 bp, Artificial Sequence
  Description of Artificial Sequence Synthetic oligonucleotide
cctacggcgtgcagtgcttcagc

SEQ ID NO: 28 - DNA, 332 bp, Artificial Sequence
  Description of Artificial Sequence Synthetic polynucleotide
acgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaag

SEQ ID NO: 29 - DNA, 295 bp, Artificial Sequence
  Description of Artificial Sequence Synthetic polynucleotide
gtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgtcgagggcgacaccctggtgaaccgcatcgagctgaagggcatcgacttcaag

SEQ ID NO: 30 - DNA, 158 bp, Artificial Sequence
  Description of Artificial Sequence Synthetic polynucleotide
tccggcgagggcgagggcgatgccacctacggcaagctgaccctgaagttcatctgcaccaccggcaagctgcccgtgccctggcccaccctcgtgaccaccctgacgagggcgacaccctggtgaaccgcatcgagctgaagggcatcgacttcaag

SEQ ID NO: 31 - DNA, 285 bp, Artificial Sequence
  Description of Artificial Sequence Synthetic polynucleotide
tccggcgagggcgagggcgatgccacctacggcaagctgaccctgaagttcatctgcaccaccggcaagctgcccgtgccctggcccaccctcgtgaacaccctgacctacgtgcagtgcttcagccgctaccccgaccacatgaagcagcacgacttcttcaagtccgccatgcccgaaggctacgtccaggagcgcaccatcttcttcaaggacgacggcaactacaagttcgagggcgacaccctggtgaaccgcatcgagctgaagggcatcgacttcaag

SEQ ID NO: 32 - DNA, 301 bp, Artificial Sequence
  Description of Artificial Sequence Synthetic polynucleotide
ccggcgagggcgagggcgatgccacctacggcaagctgaccctgaagttcatctgcaccaccggcaagctgcccgtgccctggcccaccctcgtgaccaccctgacctacgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaa

SEQ ID NO: 33 - DNA, 156 bp, Artificial Sequence
  Description of Artificial Sequence Synthetic polynucleotide
tccggcgagggcgagggcgatgccacctacggcaagctgaccctgaagttcatctgcaccaccggcaagctgcccgtgccctggcccaccctcgtgaccaccctgacctacggacaccctggtgaaccgcatcgagctgaagggcatcgacttcaa

SEQ ID NO: 34 - DNA, 197 bp, Artificial Sequence
  Description of Artificial Sequence Synthetic polynucleotide
gtaaacggccacaagttcagcgtgcagtgcttcagccgctaccccgaccacatgaagcagcacgacttcttcaagtccgccatgcccgaaggctacgtccaggagcgcaccatcttcttcaaggacgacggcaactacaagacggcaactacaagttcgagggcgacaccctggtgaaccgcatcgagctgaagggc

SEQ ID NO: 35 - DNA, 283 bp, Artificial Sequence
  Description of Artificial Sequence Synthetic polynucleotide
ccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacaccgcatcgagctgaagggcatcgacttcaa

SEQ ID NO: 36 - DNA, 175 bp, Artificial Sequence
  Description of Artificial Sequence Synthetic polynucleotide
ggccacaagttcagcgtgtccggcgagggcgagggcgatgccacctacggcaagctgaccctgaagttcatctgcaccaccggcaccatcaaggacgacggcaactacaagacccgcgccgaagttcgagggcgacaccctggtgaaccgcatcgagctgaagggcatcgacttc

SEQ ID NO: 37 - DNA, 291 bp, Artificial Sequence
  Description of Artificial Sequence Synthetic polynucleotide
taaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacctggtgaaccgcatcgagctgaagggcatcga

SEQ ID NO: 38 - DNA, 314 bp, Artificial Sequence
  Description of Artificial Sequence Synthetic polynucleotide
cacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggttcgagggcgacaccctggtgaaccgcatcgagctgaagggcatcgacttca

SEQ ID NO: 39 - DNA, 316 bp, Artificial Sequence
  Description of Artificial Sequence Synthetic polynucleotide
taaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgagggcgacaccctggtgaaccgcatcgagctgaagggcatcgacttcaa

SEQ ID NO: 40 - DNA, 318 bp, Artificial Sequence
  Description of Artificial Sequence Synthetic polynucleotide
aaacggccacaagttcagcgtgtccggcgagggcgagggcgatgccacctacggcaagctgaccctgaagttcatctgcaccaccggcaagctgcccgtgccctggcccaccctcgtgaccaccctgacctacgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcga

SEQ ID NO: 41 - DNA, 185 bp, Artificial Sequence
  Description of Artificial Sequence Synthetic polynucleotide
acggccacaagttcagcgtgtccggcgagggcgagggcgatgccacctacggcaagctgaccctgaagttcatctgcaccaccggcaagctgcccgtgccctggcccaccctcgtgaccaccctgacctacgtgaagttcgagggcgacaccctggtgaaccgcatcgagctgaagggcatcgac

SEQ ID NO: 42 - DNA, 180 bp, Artificial Sequence
  Description of Artificial Sequence Synthetic polynucleotide
aaacggccacaagttcagcgtgtccggcgagggcgagggcgatgccacctacggcaagctgaccctgaagttcatctgcaccaccggcaagctgcccgtgccctggcccaccctcgtgaccaccctgacctacgtgaagttcgagggcgacaccctggtgaaccgcatcgagctgaaggg

SEQ ID NO: 43 - DNA, 242 bp, Artificial Sequence
  Description of Artificial Sequence Synthetic polynucleotide
  modified_base (132)..(132): a, c, t, g, unknown or other
  modified_base (153)..(153): a, c, t, g, unknown or other
  modified_base (174)..(174): a, c, t, g, unknown or other
aaacggccacaagttcagcgtgtccggcgagggcgagggcgatgccacctacggcaagctgaccctgaagttcatctgcaccaccggcaagctgcccgtgccctggcccacccttccgccatgcccgaaggntacgtccaggagcgcaccatnttcttcaaggacgacggcaantacaagacccgcgccgaagttcgagggcgacaccctggtgaaccgcatcgagctgaagggcatcgact

SEQ ID NO: 44 - DNA, 175 bp, Artificial Sequence
  Description of Artificial Sequence Synthetic polynucleotide
caccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtg

SEQ ID NO: 45 - DNA, 39 bp, Artificial Sequence
  Description of Artificial Sequence Synthetic oligonucleotide
caccctgacctacgtgaagttcgagggcgacaccctggt

SEQ ID NO: 46 - DNA, 161 bp, Artificial Sequence
  Description of Artificial Sequence Synthetic polynucleotide
caccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgagggcgacaccctgg

SEQ ID NO: 47 - DNA, 145 bp, Artificial Sequence
  Description of Artificial Sequence Synthetic polynucleotide
caccctgacctcagccgctaccccgaccacatgaagcagcacgacttcttcaagtccgccatgcccgaaggctacgtccaggagcgcaccatcttcttcaaggacgacggcaactacaagacccgcgccgaggcgacaccctggt

SEQ ID NO: 48 - DNA, 153 bp, Artificial Sequence
  Description of Artificial Sequence Synthetic polynucleotide
ttattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcat

SEQ ID NO: 49 - DNA, 113 bp, Artificial Sequence
  Description of Artificial Sequence Synthetic polynucleotide
ttattatacatcggagccctgccaaaaaatcaatgtgaagcaaatcgcagcccgcctccgctctactcactggtgttcatctttggttttgtgggcaacatgctggtcatcct

SEQ ID NO: 50 - DNA, 70 bp, Artificial Sequence
  Description of Artificial Sequence Synthetic oligonucleotide
ttattatacatcggagccctgccaaaaaatcaatgtgaagcaaatcgcagcccgcatgctggtcatcctc

SEQ ID NO: 51 - RNA, 62 nt, Artificial Sequence
  Description of Artificial Sequence Synthetic oligonucleotide
  modified_base (1)..(20): a, c, u or g and this region may encompass 17-20 nucleotides wherein some positions may be absent
nnnnnnnnnnnnnnnnnnnnguuuuagagcuagaaauagcaaguuaaaauaaggcuaguccg

SEQ ID NO: 52 - RNA, 54 nt, Artificial Sequence
  Description of Artificial Sequence Synthetic oligonucleotide
  modified_base (1)..(20): a, c, u or g and this region may encompass 17-20 nucleotides wherein some positions may be absent
nnnnnnnnnnnnnnnnnnnnguuuuagagcuagaaauagcaaguuaaaauaagg

SEQ ID NO: 53 - RNA, 28 nt, Artificial Sequence
  Description of Artificial Sequence Synthetic oligonucleotide
guucacugccguauaggcagcuaagaaa

SEQ ID NO: 54 - RNA, 20 nt, Artificial Sequence
  Description of Artificial Sequence Synthetic oligonucleotide
guucacugccguauaggcag

(19) United States
(12) Patent Application Publication
(10) Pub. No.: US 2020/0165587 A1
(43) Pub. Date: May 28, 2020

(54) Multiplex Guide RNAS

(71) Applicant: The General Hospital Corporation, Boston, MA (US)

(72) Inventors: Shengdar Tsai, Charlestown, MA (US); J. Keith Joung, Winchester, MA (US)

(21) Appl. No.: 16/735,146

(22) Filed: Jan. 6, 2020

Related U.S. Application Data
(63) Continuation of application No. 15/107,550, filed on Jun. 23, 2016, now Pat. No. 10,526,589.
Application No. 15/107,550 is a continuation of application No. 14/211,117, filed on Mar. 14, 2014.
Application No. 14/211,117 is a continuation of application No. PCT/US2014/029068, filed on Mar. 14, 2014.
Application No. PCT/US2014/029068 is a continuation of application No. PCT/US2014/029304, filed on Mar. 14, 2014.
Application No. PCT/US2014/029304 is a continuation of application No. PCT/US2014/028630, filed on Mar. 14, 2014.
Application No. PCT/US2014/028630 is a continuation of application No. PCT/US2014/035162, filed on Apr. 23, 2014.
(60) Provisional application No. 61/921,007, filed on Dec. 26, 2013.
(60) Provisional application No. 61/930,782, filed on Jan. 23, 2014.

(51) Int. Cl. C12N 9/22; C12N 15/63; C12N 15/113; C07K 14/47

(52) U.S. Cl. CPC C12N 9/22; C12N 15/63; C12N 15/1136; C07K 14/4705; C12N 2310/3231; C07K 2319/40; C12N 2310/20; C12N 2310/3341; C07K 2319/80; C07K 2319/43; C12N 2310/318

(57) ABSTRACT

Methods and constructs for the multiplex expression of highly active CRISPR guide RNAs (gRNAs) from RNA Polymerase II and III promoters, optionally in mammalian cells.

### CROSS-REFERENCE TO RELATED APPLICATIONS

This application is a continuation of U.S. patent application Ser. No. 15/107,550, filed Jun. 23, 2016, which is a 371 application of PCT/US2014/056416, filed Sep. 18, 2014, which claims the benefit of U.S. Provisional Patent Applications 61/921,007, filed on Dec. 26, 2013 and 61/930,782, filed on Jan. 23, 2014; U.S. patent application Ser. No. 14/211,117, filed on Mar. 14, 2014; and International Application Nos. PCT/US2014/029068, filed on Mar. 14, 2014; PCT/US2014/028630, filed on Mar. 14, 2014; PCT/US2014/035162, filed on Apr. 23, 2014; and PCT/US2014/029304, filed on Mar. 14, 2014. All of the foregoing are incorporated herein by reference.

### FEDERALLY SPONSORED RESEARCH OR DEVELOPMENT

This invention was made with Government support under Grant No. DP1 GM105378 awarded by the National Institutes of Health. The Government has certain rights in the invention.

### TECHNICAL FIELD

Described are methods and constructs for the multiplex expression of highly active CRISPR guide RNAs (gRNAs) from RNA Polymerase II and III promoters, optionally in mammalian cells.

### BACKGROUND

The Cas9 nuclease forms the basis of a programmable RNA-guided clustered, regularly interspaced, short palindromic repeats (CRISPR)/CRISPR-associated (Cas) system (Wiedenheft et al., Nature 482, 331-338 (2012); Horvath et al., Science 327, 167-170 (2010); Terns et al., Curr Opin Microbiol 14, 321-327 (2011)) that can be used to create site-specific breaks in target DNA sequences in vitro, in mammalian cells, and in living model organisms such as zebrafish (Wang et al., Cell 153, 910-918 (2013); Shen et al., Cell Res (2013); Dicarlo et al., Nucleic Acids Res (2013); Jiang et al., Nat Biotechnol 31, 233-239 (2013); Jinek et al., Elife 2, e00471 (2013); Hwang et al., Nat Biotechnol 31, 227-229 (2013); Cong et al., Science 339, 819-823 (2013); Mali et al., Science 339, 823-826 (2013c); Cho et al., Nat Biotechnol 31, 230-232 (2013); Gratz et al., Genetics 194(4):1029-35 (2013)). A short ˜100 nt guide RNA (gRNA) complexes with Cas9 and directs the nuclease to a specific target DNA site; targeting is mediated by a sequence of at least 17-20 nucleotides (nts) at the 5′ end of the gRNA, which are designed to be complementary to and interact via simple base pair complementarity between the first 17-20 nucleotides of an engineered gRNA and the complementary strand of a target genomic DNA sequence of interest that lies next to a protospacer adjacent motif (PAM), e.g., a PAM matching the sequence NGG or NAG (Shen et al., Cell Res (2013); Dicarlo et al., Nucleic Acids Res (2013); Jiang et al., Nat Biotechnol 31, 233-239 (2013); Jinek et al., Elife 2, e00471 (2013); Hwang et al., Nat Biotechnol 31, 227-229 (2013); Cong et al., Science 339, 819-823 (2013); Mali et al., Science 339, 823-826 (2013c); Cho et al., Nat Biotechnol 31, 230-232 (2013); Jinek et al., Science 337, 816-821 (2012)). gRNAs can also direct catalytically inactivated Cas9 proteins (known as dCas9, see Jinek et al., Science 337:816-821 (2012)) that are in turn fused to effector domains (e.g., a transcriptional activation domain) see, e.g., U.S. Ser. No. 61/799,647, filed on Mar. 15, 2013, and 61/838,148, filed on Jun. 21, 2013, both of which are incorporated herein by reference. These latter systems enable RNA-guided recruitment of heterologous effector domains to genomic loci of interest.

### SUMMARY

The present invention is based, at least in part, on the discovery that Csy4, an endoribonuclease that recognizes a short RNA hairpin sequence, can be used to cleave out multiple functional gRNAs encoded on a single longer RNA transcript (produced from an RNA pol II or III promoter) in which the individual gRNAs are separated by Csy4 cleavage sites.

Thus in a first aspect the invention provides deoxyribonucleic acid (DNA) molecules comprising a plurality of sequences encoding guide RNAs (gRNAs), wherein each gRNA is flanked by at least one Csy4 cleavage sequence comprising or consisting of the sequence

(SEQ ID NO: 1)
GTTCACTGCCGTATAGGCAG
or
(SEQ ID NO: 2)
GTTCACTGCCGTATAGGCAGCTAAGAAA.

In some embodiments the DNA molecule is operably linked to a promoter sequence.

In some embodiments the DNA molecule includes two, three, or more gRNA sequences, each flanked by at least one Csy4 cleavage sequence.

In some embodiments, the promoter sequence is a RNA Polymerase II (Pol II) promoter or Pol III promoter, preferably a RNA Pol II promoter. In some embodiments the Pol II promoter is selected from the group consisting of CAG, EF1A, CAGGS, PGK, UbiC, CMV, B29, Desmin, Endoglin, FLT-1, GFPA, and SYN1 promoters.

In another aspect the invention provides a DNA molecule comprising a promoter sequence linked with one, two, three or more cassettes comprising: a sequence encoding a guide RNA, i.e., a sequence of about 100 nts, e.g., 95-300 nts, e.g., 95-105 nts for an S. Pyogenes-based system, linked to a Csy4 cleavage site, e.g., SEQ ID NO: 1 or 2.

In some embodiments the DNA molecule comprises a Pol II promoter, operably linked to a first sequence encoding a first guide RNA linked to a Csy4 cleavage site, linked to a second sequence encoding a second guide RNA linked to a Csy4 cleavage site, linked to a third sequence encoding a third guide RNA linked to a Csy4 cleavage site. In some embodiments, further guide RNAs linked to Csy4 cleavage sites are included. For example, the DNA molecule can have the following structure:

### Promoter-C4-gRNA-C4-gRNA-C4-gRNA-C4

### Promoter-C4-gRNA-C4-gRNA-C4-gRNA-C4-gRNA-C4

### Promoter-C4-gRNA-C4-gRNA-C4-gRNA-C4-gRNA-C4-gRNA C4

And so on. In this illustration C4 is a sequence encoding a Csy4 RNA cleavage site and gRNA is a sequence encoding a guide RNA.

In some embodiments, the the Cas9 sgRNA comprises the sequence:

(SEQ ID NO: 4)
(X₁₇₋₂₀)GUUUUAGAGCUAUGCUGUUUUG(X_{N});
(SEQ ID NO: 5)
(X₁₇₋₂₀)GUUUUAGAGCUA;
(SEQ ID NO: 6)
(X₁₇₋₂₀)GUUUUAGAGCUAUGCUGUUUUG;
(SEQ ID NO: 7)
(X₁₇₋₂₀)GUUUUAGAGCUAUGCU;
(SEQ ID NO: 8)
(X₁₇₋₂₀)GUUUUAGAGCUAGAAAUAGCAAGUUAAAAUAAGGCUAGUCCG
(X_{N});
(SEQ ID NO: 9)
(X₁₇₋₂₀)GUUUUAGAGCUAUGCUGAAAAGCAUAGCAAGUUAAAAUAAGGC
UAGUCCGUUAUC(X_{N});
(SEQ ID NO: 10)
(X₁₇₋₂₀)GUUUUAGAGCUAUGCUGUUUUGGAAACAAAACAGCAUAGCAAG
UUAAAAUAAGGCUAGUCCGUUAUC(X_{N});
(SEQ ID NO: 11)
(X₁₇₋₂₀)GUUUUAGAGCUAGAAAUAGCAAGUUAAAAUAAGGCUAGUCCGU
UAUCAACUUGAAAAAGUGGCACCGAGUCGGUGC(X_{N}),
(SEQ ID NO: 12)
(X₁₇₋₂₀)GUUUAAGAGCUAGAAAUAGCAAGUUUAAAUAAGGCUAGUCCGU
UAUCAACUUGAAAAAGUGGCACCGAGUCGGUGC;
(SEQ ID NO: 13)
(X₁₇₋₂₀)GUUUUAGAGCUAUGCUGGAAACAGCAUAGCAAGUUUAAAUAAG
GCUAGUCCGUUAUCAACUUGAAAAAGUGGCACCGAGUCGGUGC;
or
(SEQ ID NO: 14)
(X₁₇₋₂₀)GUUUAAGAGCUAUGCUGGAAACAGCAUAGCAAGUUUAAAUAAG
GCUAGUCCGUUAUCAACUUGAAAAAGUGGCACCGAGUCGGUGC,

wherein X₁₇₋₂₀ is a sequence complementary to the complementary strand of 17-20 consecutive nucleotides of a target sequence, preferably a target sequence immediately 5′ of a protospacer adjacent motif (PAM), and X_{N} is any sequence that does not interfere with the binding of the ribonucleic acid to Cas9. Although a sequence of X₁₇₋₂₀ is exemplified herein with the S. pyogenes Cas9 system, longer sequences can also be used, e.g., as appropriate for other systems.

In some embodiments, the DNA molecule also includes a sequence encoding a functional Csy4 enzyme.

Also provided herein are vectors comprising the DNA molecules described herein, e.g., optionally comprising a sequence encoding a functional Csy4 enzyme. Also provided herein are the multiplex transcripts produced by the DNA molecules, e.g., intact RNAs that have not yet been cleaved with Csy4.

In yet another aspect, provided herein are methods for producing a plurality of guide RNAs in a cell. The methods include expressing the DNA molecules described herein in the cell.

In some embodiments, the cell is a mammalian cell, and the cell also expresses an exogenous functional Csy4 enzyme sequence, or the method further comprises administering a functional Csy4 enzyme or nucleic acid encoding a functional Csy4 enzyme.

In another aspect the invention provides methods for altering expression of one or a plurality of target genes in a cell. The methods include expressing a DNA molecule as described herein, e.g., a DNA molecule comprising a plurality of sequences encoding guide RNAs (gRNAs), wherein each gRNA comprises a variable sequence that is complementary to at least 17-20 nts of the one or more target genes, and each gRNA is flanked by at least one Csy4 cleavage sequence comprising or consisting of the sequence

(SEQ ID NO: 1)
GTTCACTGCCGTATAGGCAG
or
(SEQ ID NO: 2)
GTTCACTGCCGTATAGGCAGCTAAGAAA.

In the present methods and compositions, the gRNA can be either a single guide RNA comprising a fused tracrRNA and crRNA, as described herein, or can include just a crRNA, and the tracrRNA can be expressed from the same or a different DNA molecule. Thus in some embodiments the DNA molecules described herein also include a sequence encoding a tracrRNA. In some embodiments, the methods include expressing in the cells a separate tracrRNA, e.g., contacting the cells with a vector or DNA molecule that expresses a tracrRNA.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic illustrating constructs used in initial multiplex experiments, as follows:
1+2: direct repeat crRNA array and Cas9, with separate tracrRNA
3+4: short crRNA array separated by Csy4 sites with Csy4, Cas9, and separate tracrRNA
5+6: full-length chimeric gRNAs separated by Csy4 site.
7: nls-FLAG tagged Cas9

FIG. 2 is a bar graph showing the results of experiments in cells expressing the constructs shown in FIG. 1. The Csy4 site+full gRNA (constructs 5 and 6) was the most efficient multiplex framework.

FIG. 3 is a schematic overview and comparison of exemplary standard and multiplex Csy4-based gRNA frameworks, and the transcripts they produce. Note that Csy4 enables the use of RNA Pol II promoters (e.g., CAG) as an alternative to U6, an RNA Pol III promoter.

FIG. 4 is a bar graph showing that Csy4 cleaves a truncated recognition site producing gRNAs with higher activity in human cells. Processing of the truncated site also leaves a clean 5′ end, effectively removing the 5′ G restriction on gRNA target sequences imposed by the U6 promoter.

FIGS. 5A-C are sequences showing evidence of 2-target multiplex editing in single human cells. Individual deletions are observed at intended site 2 or 3. Multiple deletions on the same sequence are observed for sites 2 and 3. Deletions spanning sites 2 and 3 are also observed.

FIG. 6 is a schematic showing successful multiplex expression of three gRNAs using the Csy4-based system.

FIG. 7 is a bar graph showing gRNAs excised by Csy4 from RNA Pol II-transcribed mRNA can efficiently recruit Cas9 nuclease to specific targets in human cells. In these experiments, gRNAs were expressed in longer mRNA transcripts made from the RNA Pol II CAG promoter.

### DETAILED DESCRIPTION

One potential advantage of the Cas9 system is the capability to recruit either nuclease activity or heterologous effector domains to more than one genomic locus or target site in a cell. However, such multiplex applications require the ability to efficiently express more than one gRNA in a cell. For mammalian cells, RNA polymerase III promoters (e.g., U6 promoter) have been used to express single short gRNAs. Previous attempts to express multiple gRNA components in human cells from a single transcript have not proven to be efficient.

Additional desirable capabilities for the Cas9 system would be to create inducible versions of the components and to enable tissue-specific expression of the components. RNA polymerase II promoters that are inducible and/or tissue-specific have been previously described. However, although Cas9 or dCas9 proteins could be expressed from such RNA pol II promoters, short, defined gRNAs cannot be expressed in this way as the start and stop sites of transcription from RNA pol II are imprecise. Indeed, to date, all gRNAs have been expressed from RNA polymerase III promoters, which are ideally suited for expression of short RNAs.

As demonstrated herein, Csy4, an endoribonuclease that recognizes a short RNA hairpin sequence, can be used to cleave out multiple functional gRNAs encoded on a single longer RNA transcript (produced from an RNA pol III promoter) cassette in which the individual gRNAs are separated by Csy4 cleavage sites. Functional gRNAs can be successfully cleaved from longer RNA transcripts expressed from an RNA pol II promoter.

gRNA/Csy4 Multimeric Cassettes

Thus described herein are DNA molecules that encode longer RNA transcripts, referred to herein as multimeric cassettes, which include two or more gRNA sequences, wherein each gRNA is flanked by a Csy4 cleavage sequence. The DNA molecules can also include a promoter, and can optionally include one or more other transcription regulatory sequences, e.g., enhancers, silencers, insulators, and polyA sequences. See, e.g., Xu et al., Gene. 2001 Jul. 11; 272(1-2):149-56.

Promoters

A number of promoters are known in the art that can be used in the present methods. In some embodiments, the promoter is a PolII or Pol III promoter, preferably a Pol II promoter. Various Pol II promoters have been described and can be used in the present compositions and methods, including the CAG promoter (see, e.g., Alexopoulou et al., BMC Cell Biology 9: 2, 2008; Miyazaki et al., Gene 79 (2): 269-77 (1989); Niwa et al., Gene 108 (2): 193-9 (1991); additional promoters include the EF1A, CAGGS, PGK, UbiC and CMV promoters, as well as tissue-specific promoters such as B29, Desmin, Endoglin, FLT-1, GFPA, SYN1, among others; sequences of numerous promoters are known in the art. For example, the CMV and PGK promoters can be amplified from pSicoR and pSicoR PGK respectively (Ventura et al., Proc Natl Acad Sci USA 101: 10380-10385 (2004)), the UbiC promoter can be amplified from pDSL_hpUGIH (ATCC), the CAGGS promoter can be amplified from pCAGGS (BCCM), and the EF1A promoter can be amplified from the pEF6 vector (Invitrogen). The Pol II core promoter is described in Butler and Kadonaga, Genes & Dev. 16: 2583-2592 (2002). Cleavage of the gRNAs out of a larger transcript driven by Pol II expression enables one to produce gRNAs that have any nucleotide at the 5′-most position (standard expression from a U6 or other RNA polymerase III promoter places restrictions on the identity of this nucleotide).

In some embodiments, a tissue-specific promoter is used, and a short, defined gRNA sequence can be processed out of the RNA-Pol II transcript.

A number of Pol III promoters are known in the art, including the U6 small nuclear (sn) RNA promoter, 7SK promoter, and the H1 promoter. See, e.g., Ro et al., BioTechniques, 38(4):625-627 (2005).

Guide RNAs

Cas9 nuclease can be guided to specific genomic targets of at least 17-20 nts bearing an additional proximal protospacer adjacent motif (PAM) of sequence NGG by using a single gRNA bearing at least 17-20 nts at its 5′ end that are complementary to the genomic DNA target site.

Thus, the compositions described herein can include a sequence encoding a single guide RNA (sgRNA) comprising a crRNA fused to a normally trans-encoded tracrRNA, e.g., a single Cas9 guide RNA as described in Mali et al., Science 2013 Feb. 15; 339(6121):823-6, with a sequence at the 5′ end that is complementary to 17-20 nucleotides (nts) of a target sequence immediately 5′ of a protospacer adjacent motif (PAM), e.g., NGG, NAG, or NNGG.

Methods of designing and expressing guide RNAs are known in the art. Guide RNAs generally speaking come in two different systems: 1) System 1 uses separate crRNA and tracrRNAs that function together to guide cleavage by Cas9; and 2) System 2 uses a chimeric crRNA-tracrRNA hybrid that combines the two separate guide RNAs in a single system (Jinek et al. 2012). The tracr-RNA can be variably truncated and a range of lengths has been shown to function in both the separate system (system 1) and the chimeric gRNA system (system 2). See, e.g., Jinek et al., Science 2012; 337:816-821; Mali et al., Science. 2013 Feb. 15; 339(6121):823-6; Cong et al., Science. 2013 Feb. 15; 339(6121):819-23; and Hwang and Fu et al., Nat Biotechnol. 2013 March; 31(3):227-9; Jinek et al., Elife 2, e00471 (2013)). For System 2, generally the longer length chimeric gRNAs have shown greater on-target activity but the relative specificities of the various length gRNAs currently remain undefined and therefore it may be desirable in certain instances to use shorter gRNAs. In some embodiments, the gRNAs are complementary to a region that is within about 100-800 bp upstream of the transcription start site, e.g., is within about 500 bp upstream of the transcription start site, includes the transcription start site, or within about 100-800 bp, e.g., within about 500 bp, downstream of the transcription start site. In some embodiments, vectors (e.g., plasmids) encoding more than one gRNA are used, e.g., plasmids encoding, 2, 3, 4, 5, or more gRNAs directed to different sites in the same region of the target gene. Additional guide RNAs, and methods of increasing the specificity of genome editing, are described in Provisional Patent Application Ser. No. 61/838,178, entitled INCREASING SPECIFICITY FOR RNA-GUIDED GENOME EDITING

In some embodiments, the gRNA comprises or consists of the sequence:

(SEQ ID NO: 4)
(X₁₇₋₂₀)GUUUUAGAGCUAUGCUGUUUUG(X_{N});
(SEQ ID NO: 5)
(X₁₇₋₂₀)GUUUUAGAGCUA;
(SEQ ID NO: 6)
(X₁₇₋₂₀)GUUUUAGAGCUAUGCUGUUUUG;
(SEQ ID NO: 7)
(X₁₇₋₂₀)GUUUUAGAGCUAUGCU;
(SEQ ID NO: 8)
(X₁₇₋₂₀)GUUUUAGAGCUAGAAAUAGCAAGUUAAAAUAAGGCUAGUCCG
(X_{N});
(SEQ ID NO: 9)
(X₁₇₋₂₀)GUUUUAGAGCUAUGCUGAAAAGCAUAGCAAGUUAAAAUAAGGC
UAGUCCGUUAUC(X_{N});
(SEQ ID NO: 10)
(X₁₇₋₂₀)GUUUUAGAGCUAUGCUGUUUUGGAAACAAAACAGCAUAGCAAG
UUAAAAUAAGGCUAGUCCGUUAUC(X_{N});
(SEQ ID NO: 11)
(X₁₇₋₂₀)GUUUUAGAGCUAGAAAUAGCAAGUUAAAAUAAGGCUAGUCCGU
UAUCAACUUGAAAAAGUGGCACCGAGUCGGUGC(X_{N}),
(SEQ ID NO: 12)
(X₁₇₋₂₀)GUUUAAGAGCUAGAAAUAGCAAGUUUAAAUAAGGCUAGUCCGU
UAUCAACUUGAAAAAGUGGCACCGAGUCGGUGC;
(SEQ ID NO: 13)
(X₁₇₋₂₀)GUUUUAGAGCUAUGCUGGAAACAGCAUAGCAAGUUUAAAUAAG
GCUAGUCCGUUAUCAACUUGAAAAAGUGGCACCGAGUCGGUGC;
or
(SEQ ID NO: 14)
(X₁₇₋₂₀)GUUUAAGAGCUAUGCUGGAAACAGCAUAGCAAGUUUAAAUAAG
GCUAGUCCGUUAUCAACUUGAAAAAGUGGCACCGAGUCGGUGC,

wherein X₁₇₋₂₀ is a sequence complementary to the complementary strand of at least 17-20 consecutive nucleotides of a target sequence (though in some embodiments this complementarity region may be longer than 20 nts, e.g., 21, 22, 23, 24, 25, 26, 27, 28, 29, or 30 or more nts, e.g., 17-30 nts), preferably a target sequence immediately 5′ of a protospacer adjacent motif (PAM), e.g., NGG NAG or NNGG X_{N} is any sequence, wherein N (in the RNA) can be 0-300 or 0-200, e.g., 0-100, 0-50, or 0-20, that does not interfere with the binding of the ribonucleic acid to Cas9. In some embodiments the RNA includes one or more U, e.g., 1 to 8 or more Us (e.g., U, UU, UUU, UUUU, UUUUU, UUUUUU, UUUUUUU, UUUUUUUU) at the 3′ end of the molecule, as a result of the optional presence of one or more Ts used as a termination signal to terminate RNA PolIII transcription. In some embodiments the RNA includes one or more, e.g., up to 3, e.g., one, two, or three, or more additional nucleotides at the 5′ end of the RNA molecule that is not complementary to the target sequence. Optionally, one or more of the RNA nucleotides is modified, e.g., locked (2′-O-4′-C methylene bridge), is 5′-methylcytidine, is 2′-O-methyl-pseudouridine, or in which the ribose phosphate backbone has been replaced by a polyamide chain, e.g., one or more of the nucleotides within the sequence X₁₇₋₂₀, one or more of the nucleotides within the sequence X_{N}, or one or more of the nucleotides within any sequence of the gRNA

For example, in some embodiments the chimeric guide RNAs described in Jinek et al. (Science. 337(6096):816-21 (2012)) can be used, e.g.,

(SEQ ID NO: 8)
(X₁₇₋₂₀₎GUUUUAGAGCUAGAAAUAGCAAGUUAAAAUAAGGCUAGUCCG;
(SEQ ID NO: 9)
(X₁₇₋₂₀₎GUUUUAGAGCUAGAAAUAGCAAGUUAAAAUAAGG;

in some embodiments, the sgRNA bearing a 5′-terminal 17-20-nucleotide sequence complementary to the target DNA sequence, and a 42-nucleotide 3′-terminal stem loop structure required for Cas9 binding described in Jinek et al., Elife. 2:e00471 (2013), e.g., (X₁₇₋₂₀)GUUUUAGAGCUAGAAAUAGCAAGUUAAAAUAAGGCUAGUCCG (SEQ ID NO:8) are used.

In some embodiments, the guide RNA includes one or more Adenine (A) or Uracil (U) nucleotides on the 3′ end.

Although the examples described herein utilize a single gRNA, the methods can also be used with dual gRNAs (e.g., the crRNA and tracrRNA found in naturally occurring systems). In this case, a single tracrRNA would be used in conjunction with multiple different crRNAs expressed using the present system, e.g., the following (note that for RNAs, T's are understood herein to be U's):

crRNA sequence: X₁₇₋₂₀-GTTTTAGAGCTAGAAA (SEQ ID NO: 15)

tracrRNA sequence: TAGCAAGTTAAAATAAGGCTAGTCCGTTATCAACTTGAAAAAGTGGCACCGAGT CGGTGC (SEQ ID NO: 16). In this case, the crRNA is used as the guide RNA in the methods and molecules described herein, and the tracrRNA can be expressed from the same or a different DNA molecule.

Furthermore, although guide RNAs having a sequence of 17-20 nucleotides of complementarity are exemplified herein, in some embodiments longer sequences can be used, e.g., 21, 22, 23, 24, 25, 26, 27, 28, 29, or 30 or more nts, e.g., 17-30 nts in place of 17-20 nts.

Csy4 Cleavage Sequences

In the methods and compositions described herein, a Csy4 cleavage sequence is inserted into the DNA molecule such that each guide RNA is flanked by a cleavage sequence, with one or at least one cleavage sequence between each guide RNA. Exemplary Csy4 cleavage sequences include GTTCACTGCCGTATAGGCAG (truncated 20 nt) (SEQ ID NO: 1) and GTTCACTGCCGTATAGGCAGCTAAGAAA (full 28 nt) (SEQ ID NO:2). As demonstrated herein, use of the truncated Csy4 cleavage site (SEQ ID NO: 1) is more efficient in human cells than use of the standard site. To the best of the present inventors' knowledge, this is the first demonstration of Csy4 activity being utilized in human cells.

Functional Csy4 Enzyme Sequences

In the methods described herein, a functional Csy4 enzyme that is capable of cleaving the transcripts at the Csy4 cleavage sites, is also expressed in the cell.

Exemplary Csy4 sequences from Csy4 homologues from Pseudomonas aeruginosa UCBPP-PA14 (Pa14), Yersinia pestis AAM85295 (Yp), Escherichia coli UTI89 (Ec89), Dichelobacter nodosus VCS1703A (Dn), Acinetobacter baumannii AB0057 (Ab), Moritella sp. PE36 (MP1, MP01), Shewanella sp. W3-18-1 (SW), Pasteurella multocida subsp. multocida Pm70 (Pm), Pectobacterium wasabiae (Pw), and Dickeya dadantii Ech703 (Dd) are set forth in Fig. S6 of Haurwitz et al., Science 329(5997): 1355-1358 (2010). In preferred embodiments, the Csy4 is from Pseudomonas aeruginosa.

In some embodiments, the Csy4 is also used to covalently link heterologous effector domains to the gRNAs. Csy4 is believed to be a single-turnover enzyme and remains bound to its target hairpin sequence after cleavage (Sternberg et al., RNA. 2012 April; 18(4):661-72). Csy4 is thus expected to remain bound to the 3′ end of each cleaved gRNA. Since as demonstrated herein the cleaved gRNAs appear to be functional in human cells, the presence of this Csy4 protein on the 3′ end of the gRNA does not appear to affect the ability of the gRNA to complex with and direct Cas9 activity. Thus it is presumed that these gRNA-Csy4 fusions would also be able to direct Cas9 mutants that bear mutations that inactivate its catalytic nuclease activity (dCas9 proteins). Therefore, one could fuse heterologous functional domains (HFD) to Csy4 (Csy4-HFD), and a dCas9:sgRNA:Csy4-HFD complex could then direct such domains to a specific genomic locus. Examples of such HFD could include other nuclease domains such as that from FokI, transcriptional activator or repressor domains, or other domains that modify histones or DNA methylation status.

The Csy4-HFD are created by fusing a heterologous functional domain (e.g., a transcriptional activation domain, e.g., from VP64 or NF-κB p65), to the N-terminus or C-terminus of a Csy4 protein, with or without an intervening linker, e.g., a linker of about 5-20 or 13-18 nucleotides. The transcriptional activation domains can be fused on the N or C terminus of the Csy4. In addition to transcriptional activation domains, other heterologous functional domains (e.g., transcriptional repressors (e.g., KRAB, SID, and others) or silencers such as Heterochromatin Protein 1 (HP1, also known as swi6), e.g., HP1α or HP1β; proteins or peptides that could recruit long non-coding RNAs (lncRNAs) fused to a fixed RNA binding sequence such as those bound by the MS2 coat protein, endoribonuclease Csy4, or the lambda N protein; enzymes that modify the methylation state of DNA (e.g., DNA methyltransferase (DNMT) or TET proteins); or enzymes that modify histone subunits (e.g., histone acetyltransferases (HAT), histone deacetylases (HDAC), histone methyltransferases (e.g., for methylation of lysine or arginine residues) or histone demethylases (e.g., for demethylation of lysine or arginine residues)) as are known in the art can also be used. A number of sequences for such domains are known in the art, e.g., a domain that catalyzes hydroxylation of methylated cytosines in DNA. Exemplary proteins include the Ten-Eleven-Translocation (TET)1-3 family, enzymes that converts 5-methylcytosine (5-mC) to 5-hydroxymethylcytosine (5-hmC) in DNA. See, e.g., WO/2014/144761.

Sequences for human TET1-3 are known in the art and are shown in the following table:

| | GenBank Accession Nos. | |
|---|---|---|
| Gene | Amino Acid | Nucleic Acid |
| TET1 | NP_085128.2 | NM_030625.2 |
| TET2* | NP_001120680.1 (var 1) | NM_001127208.2 |
| | NP_060098.3 (var 2) | NM_017628.4 |
| TET3 | NP_659430.1 | NM_144993.1 |

*Variant (1) represents the longer transcript and encodes the longer isoform (a).
Variant (2) differs in the 5′UTR and in the 3′UTR and coding sequence compared to variant 1.
The resulting isoform (b) is shorter and has a distinct C-terminus compared to isoform a.

In some embodiments, all or part of the full-length sequence of the catalytic domain can be included, e.g., a catalytic module comprising the cysteine-rich extension and the 2OGFeDO domain encoded by 7 highly conserved exons, e.g., the Tet1 catalytic domain comprising amino acids 1580-2052, Tet2 comprising amino acids 1290-1905 and Tet3 comprising amino acids 966-1678. See, e.g., FIG. 1 of Iyer et al., Cell Cycle. 2009 Jun. 1; 8(11):1698-710. Epub 2009 Jun. 27, for an alignment illustrating the key catalytic residues in all three Tet proteins, and the supplementary materials thereof (available at ftp site ftp.ncbi.nih.gov/pub/aravind/DONS/supplementary_material_DONS.html) for full length sequences (see, e.g., seq 2c); in some embodiments, the sequence includes amino acids 1418-2136 of Tet1 or the corresponding region in Tet2/3.

Other catalytic modules can be, e.g., from the proteins identified in Iyer et al., 2009.

In some embodiments, the fusion proteins include a linker between the Csy4 and the heterologous functional domains. Linkers that can be used in these fusion proteins (or between fusion proteins in a concatenated structure) can include any sequence that does not interfere with the function of the fusion proteins. In preferred embodiments, the linkers are short, e.g., 2-20 amino acids, and are typically flexible (i.e., comprising amino acids with a high degree of freedom such as glycine, alanine, and serine). In some embodiments, the linker comprises one or more units consisting of GGGS (SEQ ID NO:3) or GGGGS (SEQ ID NO:17), e.g., two, three, four, or more repeats of the GGGS (SEQ ID NO:3) or GGGGS (SEQ ID NO: 17) unit. Other linker sequences can also be used, e.g., GGS, GGSG (SEQ ID NO:22), SGSETPGTSESA (SEQ ID NO:23), SGSETPGTSESATPES (SEQ ID NO:24), or SGSETPGTSESATPEGGSGGS (SEQ ID NO:25).

Cas9

In the methods described herein, Cas9 is also expressed in the cells. A number of bacteria express Cas9 protein variants. The Cas9 from Streptococcus pyogenes is presently the most commonly used; some of the other Cas9 proteins have high levels of sequence identity with the S. pyogenes Cas9 and use the same guide RNAs. Others are more diverse, use different gRNAs, and recognize different PAM sequences as well (the 2-5 nucleotide sequence specified by the protein which is adjacent to the sequence specified by the RNA). Chylinski et al. classified Cas9 proteins from a large group of bacteria (RNA Biology 10:5, 1-12; 2013), and a large number of Cas9 proteins are listed in supplementary FIG. 1 and supplementary table 1 thereof, which are incorporated by reference herein. The constructs and methods described herein can include the use of any of those Cas9 proteins, and their corresponding guide RNAs or other guide RNAs that are compatible. The Cas9 from Streptococcus thermophilus LMD-9 CRISPR1 system has also been shown to function in human cells in Cong et al (Science 339, 819 (2013)). Additionally, Jinek et al. showed in vitro that Cas9 orthologs from S. thermophilus and L. innocua, (but not from N. meningitidis or C. jejuni, which likely use a different guide RNA), can be guided by a dual S. pyogenes gRNA to cleave target plasmid DNA, albeit with slightly decreased efficiency.

In some embodiments, the present system utilizes the Cas9 protein from S. pyogenes, either as encoded in bacteria or codon-optimized for expression in mammalian cells. An exemplary sequence of Streptococcus pyogenes Cas9 (residues 1-1368) fused to an HA epitope (amino acid sequence DAYPYDVPDYASL (SEQ ID NO: 18)) and a nuclear localization signal (amino acid sequence PKKKRKVEDPKKKRKVD (SEQ ID NO: 19)) is as follows:

(SEQ ID NO: 20)
MDKKYSIGLDIGINSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLIP
NFKSNFDLAEDAKLQLSKDTYDDDLDNLLAQIGDQYADLFLAAKNLSDAI
LLSDILRVNTEITKAPLSASMIKRYDEHHQDLILLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMINFDK
NLPNEKVLPKHSLLYEYFTVYNELTKVKYVTEGMRKPAFLSGEQKKAIVD
LLFKINRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGDAYPYDVPDYASLGSGSPKKKRKVEDPKKKRKV
D

See Jinek et al, 2013, supra.

In some embodiment, a Cas9 sequence is used that contains either of the D10A and H840A mutations to render the nuclease a nickase, or both the D10A and H840A mutations to render the nuclease portion of the protein catalytically inactive. The sequence of a catalytically inactive S. pyogenes Cas9 (dCas9) that can be used in the methods and compositions described herein is as follows; the mutations are in bold and underlined.

```
(SEQ ID NO: 21)
                10 20 30 40
MDKKYSIGLA IGTNSVGWAV ITDEYKVPSK KFKVLGNTDR
                50 60 70 80
HSIKKNLIGA LLFDSGETAE ATRLKRTARR RYTRRKNRIC
                90 100 110 120
YLQEIFSNEM AKVDDSFFHR LEESFLVEED KKHERHPIFG
              130 140 150 160
NIVDEVAYHE KYPTIYHLRK KLVDSTDKAD LRLIYLALAH
              170 180 190 200
MIKFRGHFLI EGDLNPDNSD VDKLFIQLVQ TYNQLFEENP
              210 220 230 240
INASGVDAKA ILSARLSKSR RLENLIAQLP GEKKNGLFGN
              250 260 270 280
LIALSLGLTP NFKSNFDLAE DAKLQLSKDT YDDDLDNLLA
              290 300 310 320
QIGDQYADLF LAAKNLSDAI LLSDILRVNT EITKAPLSAS
              330 340 350 360
MIKRYDEHHQ DLTLLKALVR QQLPEKYKEI FFDQSKNGYA
              370 380 390 400
GYIDGGASQE EFYKFIKPIL EKMDGTEELL VKLNREDLLR
              410 420 430 440
KQRTFDNGSI PHQIHLGELH AILRRQEDFY PFLKDNREKI
              450 460 470 480
EKILTFRIPY YVGPLARGNS RFAWMTRKSE ETITPWNFEE
              490 500 510 520
VVDKGASAQS FIERMTNFDK NLPNEKVLPK HSLLYEYFTV
              530 540 550 560
YNELTKVKYV TEGMRKPAFL SGEQKKAIVD LLFKTNRKVT
              570 580 590 600
VKQLKEDYFK KIECFDSVEI SGVEDRFNAS LGTYHDLLKI
              610 620 630 640
IKDKDFLDNE ENEDILEDIV LTLTLFEDRE MIEERLKTYA
              650 660 670 680
HLFDDKVMKQ LKRRRYTGWG RLSRKLINGI RDKQSGKTIL
              690 700 710 720
DFLKSDGFAN RNFMQLIHDD SLTFKEDIQK AQVSGQGDSL
              730 740 750 760
HEHIANLAGS PAIKKGILQT VKVVDELVKV MGRHKPENIV
              770 780 790 800
IEMARENQTT QKGQKNSRER MKRIEEGIKE LGSQILKEHP
              810 820 830 840
VENTQLQNEK LYLYYLQNGR DMYVDQELDI NRLSDYDVDA
              850 860 870 880
IVPQSFLKDD SIDNKVLTRS DKNRGKSDNV PSEEVVKKMK
              890 900 910 920
NYWRQLLNAK LITQRKFDNL TKAERGGLSE LDKAGFIKRQ
              930 940 950 960
LVETRQITKH VAQILDSRMN TKYDENDKLI REVKVITLKS
              970 980 990 1000
KLVSDFRKDF QFYKVREINN YHHAHDAYLN AVVGTALIKK
            1010 1020 1030 1040
YPKLESEFVY GDYKVYDVRK MIAKSEQEIG KATAKYFFYS
            1050 1060 1070 1080
NIMNFFKTEI TLANGEIRKR PLIETNGETG EIVWDKGRDF
            1090 1100 1110 1120
ATVRKVLSMP QVNIVKKTEV QTGGFSKESI LPKRNSDKLI
            1130 1140 1150 1160
ARKKDWDPKK YGGFDSPTVA YSVLVVAKVE KGKSKKLKSV
            1170 1180 1190 1200
KELLGITIME RSSFEKNPID FLEAKGYKEV KKDLIIKLPK
            1210 1220 1230 1240
YSLFELENGR KRMLASAGEL QKGNELALPS KYVNFLYLAS
            1250 1260 1270 1280
HYEKLKGSPE DNEQKQLFVE QHKHYLDEII EQISEFSKRV
            1290 1300 1310 1320
ILADANLDKV LSAYNKHRDK PIREQAENII HLFTLTNLGA
            1330 1340 1350 1360
PAAFKYFDTT IDRKRYTSTK EVLDATLIHQ SITGLYETRI
DLSQLGGD
```

See, e.g., Mali et al., 2013, supra; and Jinek et al., 2012, supra. Alternatively, the Cas9 can be a dCas9-heterofunctional domain fusion (dCas9-HFD) as described in U.S. Provisional Patent Application entitled RNA-GUIDED TARGETING OF GENETIC AND EPIGENOMIC REGULATORY PROTEINS TO SPECIFIC GENOMIC LOCI, filed on Jun. 21, 2013 and assigned Ser. No. 61/838,148, and in PCT/US2014/027335.

The Cas9 can be expressed from an expression vector, as described herein, e.g., an extrachromosomal plasmid or viral vector comprising a sequence encoding Cas9, e.g., a Cas9 cDNA or gene; can be expressed from an exogenous Cas9 cDNA or gene that has integrated into the genome of the cell; an mRNA encoding Cas9; the actual Cas9 protein itself; or, in the case of non-mammalian cells, can be an exogenous Cas9.

Expression Systems

Nucleic acid molecules comprising expression vectors can be used, e.g., for in vivo or in vitro expression of the Csy4/guide RNA constructs described herein. Vectors for expressing multiple gRNAs (potentially in an inducible or tissue-/cell-type specific fashion) can be used for research and therapeutic applications.

In order to use the fusion proteins and multimeric guide RNA cassettes described herein, it may be desirable to express them from a nucleic acid that encodes them. This can be performed in a variety of ways. For example, a nucleic acid encoding a guide RNA cassette or Csy4 or Cas9 protein can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the fusion protein or for production of the fusion protein. The nucleic acid encoding the guide RNA or fusion protein can also be cloned into an expression vector, for administration to a plant cell, animal cell, preferably a mammalian cell or a human cell, fungal cell, bacterial cell, or protozoan cell.

To obtain expression, a sequence encoding a guide RNA or fusion protein is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

A number of suitable vectors are known in the art, e.g., viral vectors including recombinant retroviruses, lentivirus, adenovirus, adeno-associated virus, and herpes simplex virus 1, adenovirus-derived vectors, or recombinant bacterial or eukaryotic plasmids. For example, the expression construct can include: a coding region; a promoter sequence, e.g., a promoter sequence that restricts expression to a selected cell type, a conditional promoter, or a strong general promoter; an enhancer sequence; untranslated regulatory sequences, e.g., a 5′untranslated region (UTR), a 3′UTR; a polyadenylation site; and/or an insulator sequence. Such sequences are known in the art, and the skilled artisan would be able to select suitable sequences. See, e.g., Current Protocols in Molecular Biology, Ausubel, F. M. et al. (eds.) Greene Publishing Associates, (1989), Sections 9.10-9.14 and other standard laboratory manuals. In some embodiments, expression can be restricted to a particular cell type, using a tissue-specific promoter as is known in the art.

As described above, the vectors for expressing the guide RNAs can include RNA Pol II or Pol III promoters to drive expression of the guide RNAs. These human promoters allow for expression of gRNAs in mammalian cells following plasmid transfection. Alternatively, a T7 promoter may be used, e.g., for in vitro transcription, and the RNA can be transcribed in vitro and purified. The promoter used to direct expression of the nucleic acid depends on the particular application. For example, a strong constitutive promoter is typically used for expression and purification of fusion proteins. In contrast, when the fusion protein is to be administered in vivo for gene regulation, either a constitutive or an inducible promoter can be used, depending on the particular use of the fusion protein. In addition, a preferred promoter for administration of the fusion protein can be a weak promoter, such as HSV TK or a promoter having similar activity. The promoter can also include elements that are responsive to transactivation, e.g., hypoxia response elements, Gal4 response elements, lac repressor response element, and small molecule control systems such as tetracycline-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88:1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the fusion protein, and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the fusion protein, e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ. A preferred tag-fusion protein is the maltose binding protein (MBP). Such tag-fusion proteins can be used for purification of the engineered TALE repeat protein. Epitope tags can also be added to recombinant proteins to provide convenient methods of isolation, for monitoring expression, and for monitoring cellular and subcellular localization, e.g., c-myc or FLAG

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the fusion protein encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

In some embodiments, the Cas9 or Csy4 protein includes a nuclear localization domain which provides for the protein to be translocated to the nucleus. Several nuclear localization sequences (NLS) are known, and any suitable NLS can be used. For example, many NLSs have a plurality of basic amino acids, referred to as a bipartite basic repeats (reviewed in Garcia-Bustos et al, 1991, Biochim. Biophys. Acta, 1071:83-101). An NLS containing bipartite basic repeats can be placed in any portion of chimeric protein and results in the chimeric protein being localized inside the nucleus. In preferred embodiments a nuclear localization domain is incorporated into the final fusion protein, as the ultimate functions of the fusion proteins described herein will typically require the proteins to be localized in the nucleus. However, it may not be necessary to add a separate nuclear localization domain in cases where the protein has intrinsic nuclear translocation function.

The present invention includes the vectors and cells comprising the vectors.

Libraries

Also provided herein are combinatorial libraries of gRNAs, e.g., in inducible, tissue- or cell-type specific multiplex vectors for research applications, e.g., for screening for potential drug targets or to define interactions at a genetic level.

Methods of Use

The methods described can include expressing in a cell, or contacting the cell with, the multimeric cassettes as described herein, plus a nuclease that can be guided by the shortened gRNAs, e.g., a Cas9 nuclease as described above, and a Csy4 nuclease, as described above.

The described system is a useful and versatile tool for modifying the expression of multiple endogenous genes simultaneously, or for targeting multiple parts of a single gene. Current methods for achieving this require the use of a separate gRNA-encoding transcript for each site to be targeted. Separate gRNAs are not optimal for multiplex genome editing of cell populations as it is impossible to guarantee that each cell will express each gRNA; with multiple transcripts, cells get a complex and non-uniform random mixture of gRNAs. The present system, however, allows expression of multiple gRNAs from a single transcript, which allows targeting of multiple sites in the genome by expression of multiple gRNAs. Furthermore, with a single-transcript system, each cell should express all gRNAs with similar stoichiometry. This system could therefore easily be used to simultaneously alter expression of a large number of genes or to recruit multiple Cas9s or HFDs to a single gene, promoter, or enhancer. This capability will have broad utility, e.g., for basic biological research, where it can be used to study gene function and to manipulate the expression of multiple genes in a single pathway, and in synthetic biology, where it will enable researchers to create circuits in cell that are responsive to multiple input signals. The relative ease with which this technology can be implemented and adapted to multiplexing will make it a broadly useful technology with many wide-ranging applications.

The methods described herein include contacting cells with a nucleic acid encoding the multimeric gRNA cassettes described herein directed to one or more genes, and nucleic acids encoding Csy4 and Cas9, to thereby modulate expression of the one or more genes.

### EXAMPLES

The invention is further described in the following examples, which do not limit the scope of the invention described in the claims.

### Example 1. Multiplex Editing with CRISPR/Cas9

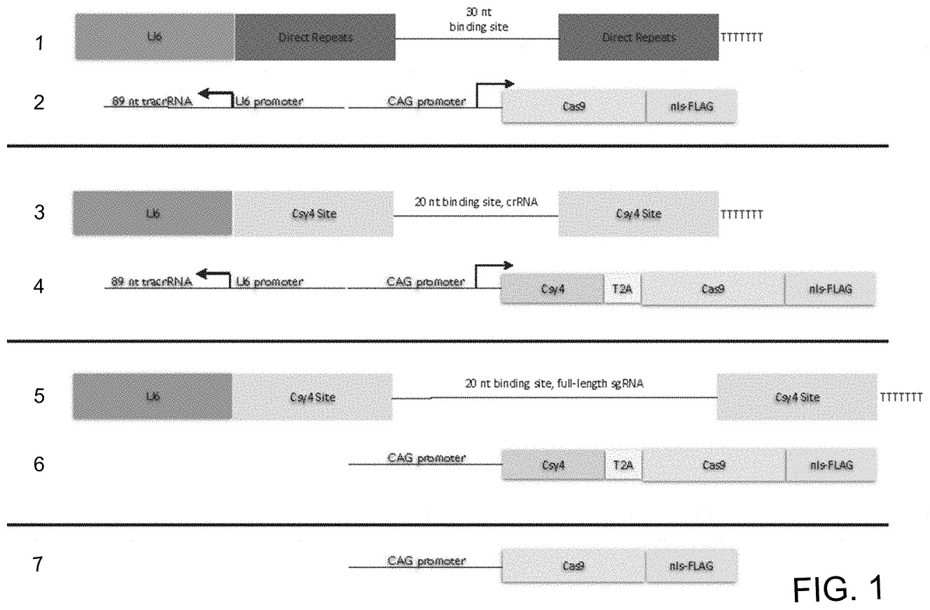

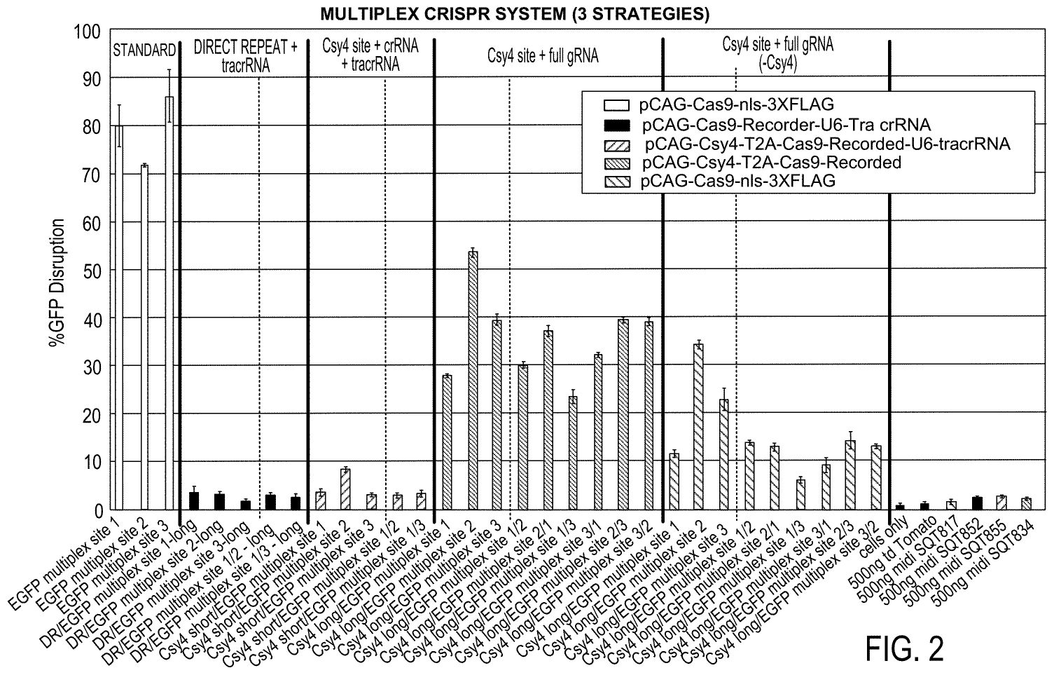

Three strategies were tried with the objective of making multiplex edits with CRISPR/Cas9 from arrays of either crRNAs or sgRNAs expressed from a single transcript, as follows:
1. direct-repeat flanked crRNA array and Cas9, with separate tracrRNA
2. short crRNA array separated by Csy4 sites, expressed with Csy4, Cas9, and separate tracrRNA
3. full-length single guide RNAs (sgRNAs) separated by Csy4 sites
   Each set of constructs (illustrated in FIG. 1) was tested for the ability to efficiently disrupt EGFP in a U2OS-EGFP disruption assay. The results are shown in FIG. 2. Constructs designed using strategies 1 and 2 exhibited the lowest activity in the EGFP-disruption assay even for single targets; therefore further experiments (described below) focused on optimizing strategy 3.

### Example 2. Multiplex Expression of Highly Active CRISPR Guide RNAs from RNA Polymerase II and III Promoters in Mammalian Cells

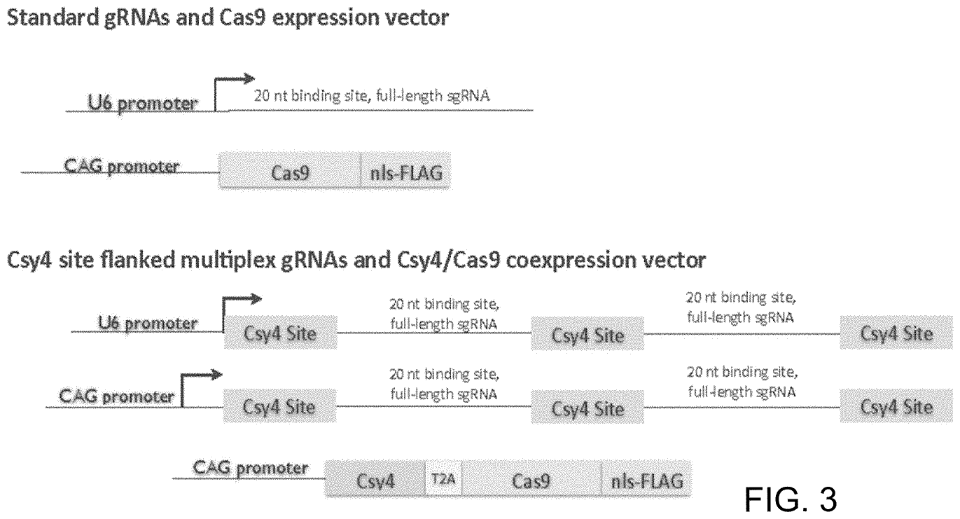

A schematic overview of an exemplary strategy for cleaving gRNAs out from longer transcripts using the Csy4 nuclease is shown in FIG. 3. In initial experiments to demonstrate proof-of-concept, two versions of the Csy4-cleaved RNA hairpin site were tested for cleavage in human cells. To do this, gRNAs flanked by one of two Csy4 cleavage sites were expressed:

1.
(SEQ ID NO: 2)
GTTCACTGCCGTATAGGCAGCTAAGAAA (full 28 nt)
2.
(SEQ ID NO: 1)
GTTCACTGCCGTATAGGCAG (truncated 20 nt)

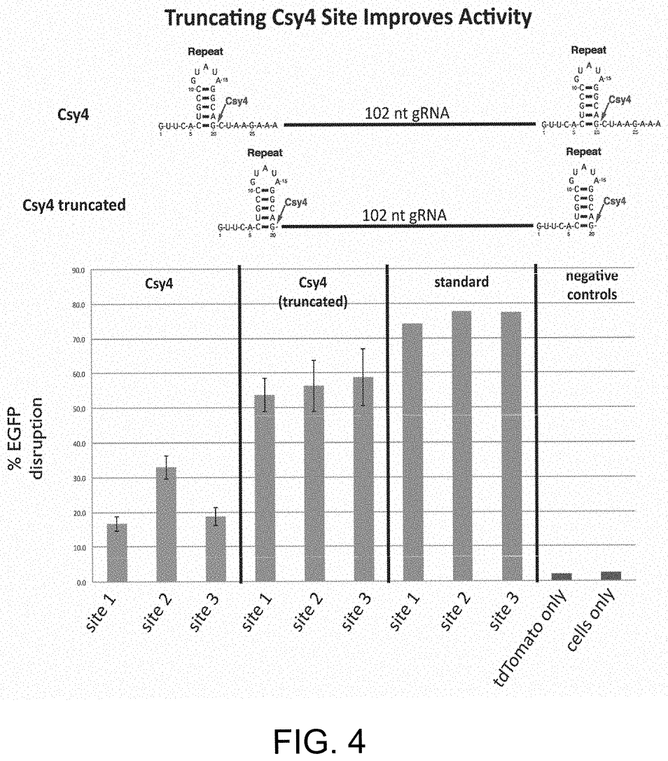

The results showed that gRNAs flanked on their 5′ and 3′ ends with the truncated 20 nt sequence were more active in mammalian cells than those flanked by the longer 28 nt sequence (FIG. 4). To the best of the present inventors' knowledge, this is the first demonstration that Csy4 nuclease can be used to process RNA transcripts in live human cells. One important additional advantage of the 20 nt truncated site is that, unlike the longer 28 nt sequence, it does not leave any additional nucleotides on the 5′ end of a gRNA processed from the longer transcript (FIG. 4). This enables expression of gRNAs that have ANY desired nucleotide at the 5′-most position. This is an improvement relative to expression of gRNAs from RNA polymerase III promoters which have a requirement for specific nucleotide(s) at the 5′-most position.

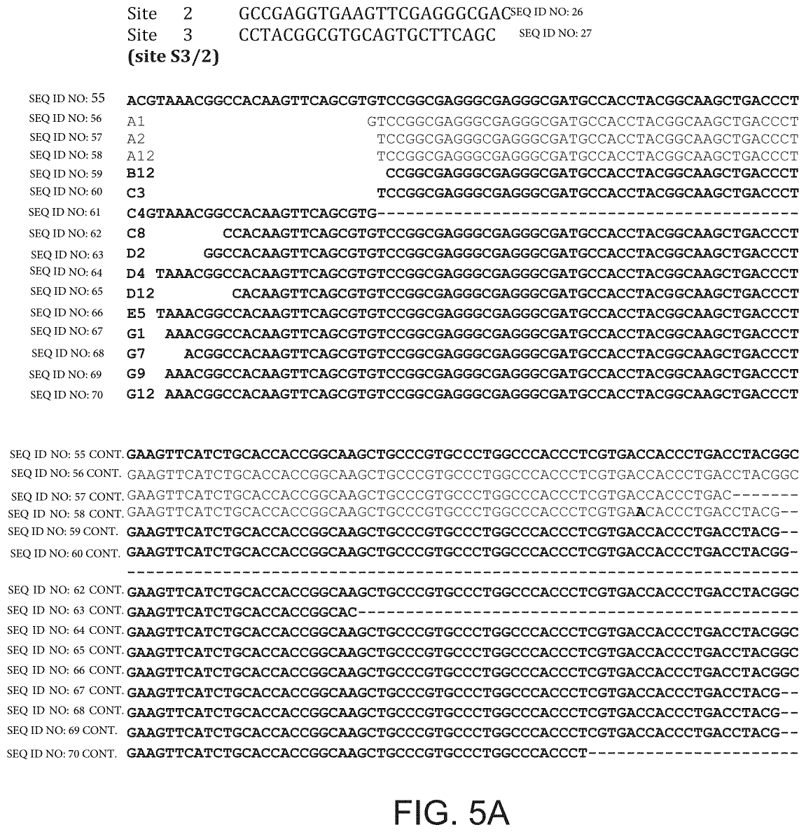

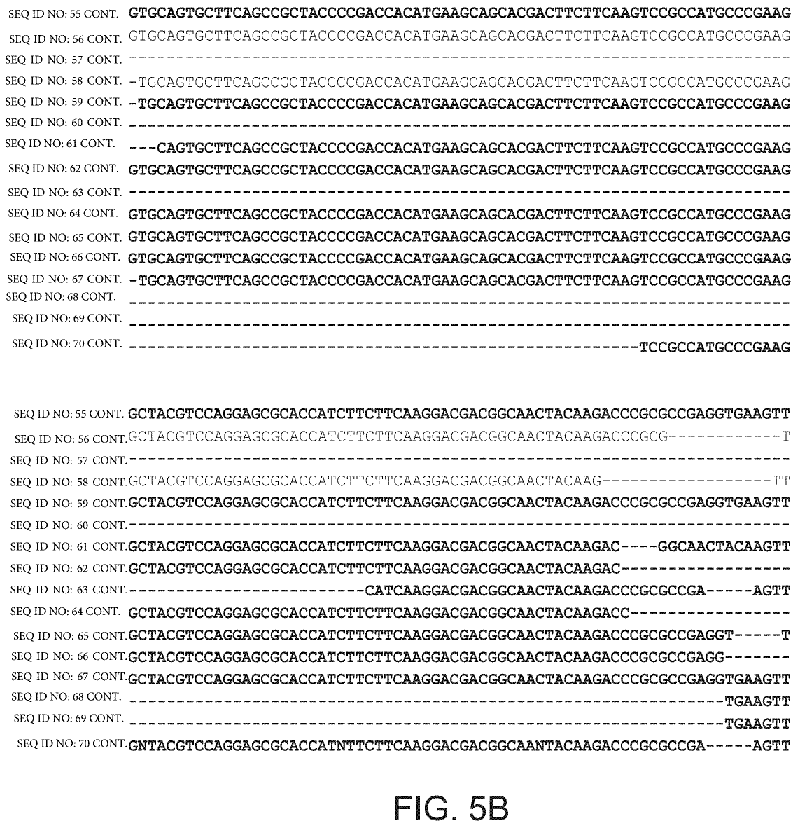

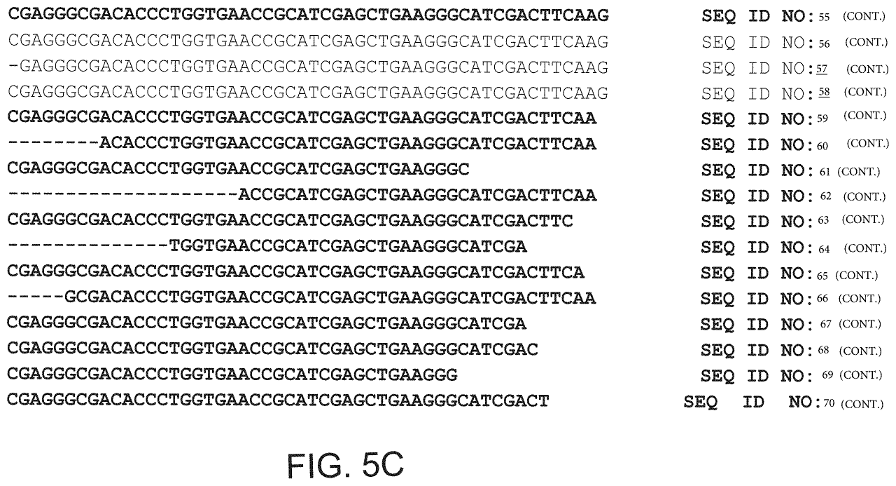

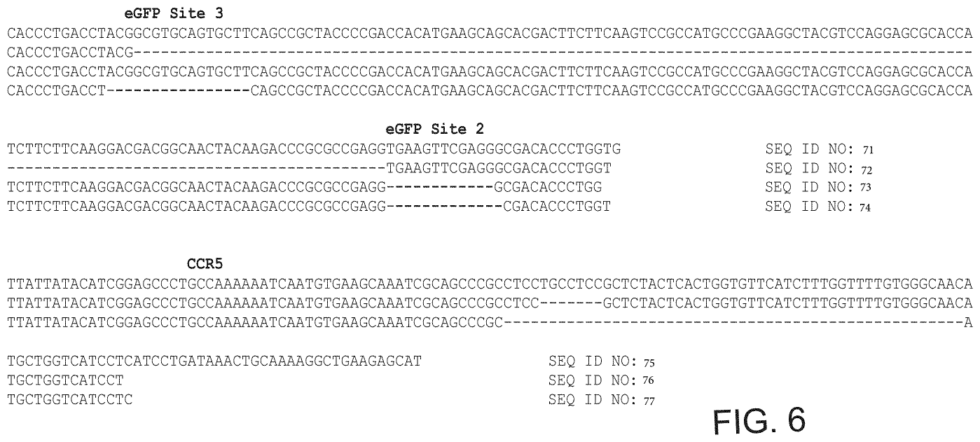

Using this Csy4-based system, the efficient expression of two and three different gRNAs (FIGS. 5 and 6) was demonstrated. gRNAs simultaneously expressed using this approach induced alterations at the expected sites in human cells.

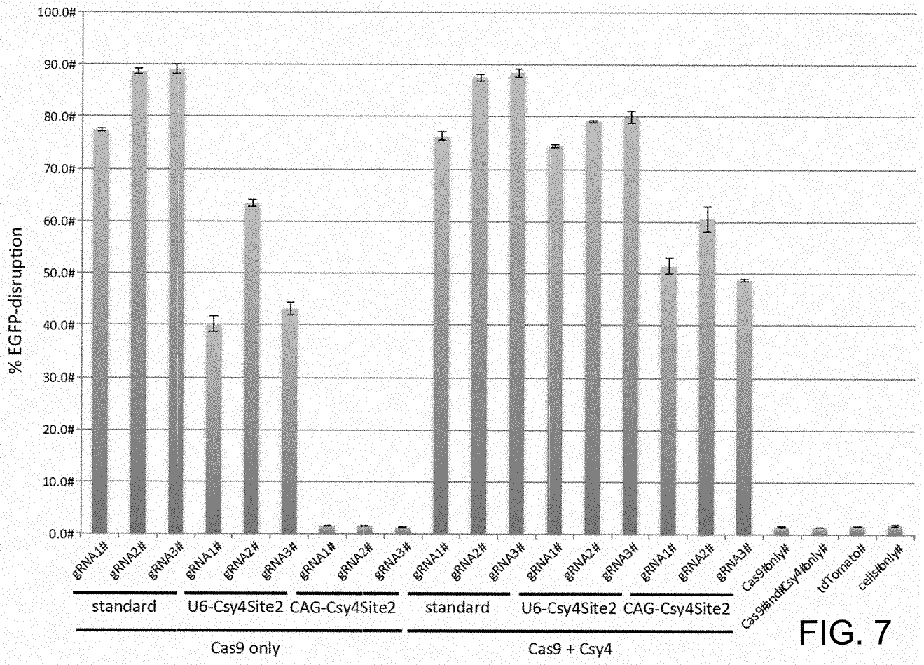

These results also demonstrated that this Csy4-based strategy could be used with gRNAs encoded on a longer mRNA produced by an RNA Pol II promoter (FIG. 7). In these experiments, one of three different single gRNAs flanked by the truncated Csy4 sites was encoded on an mRNA produced by the CAG promoter (an RNA Pol II promoter). As shown in FIG. 7, all three of these constructs could produce functional gRNA that could direct Cas9 nuclease in human cells but only in the presence of Csy4. The level of targeted Cas9 activity observed was comparable to (albeit somewhat lower than) what is observed when these gRNAs are expressed singly using a standard RNA Pol III promoter or as a Csy4-flanked transcript from an RNA Pol III promoter (FIG. 7).

In summary, the present results demonstrated that: 1) up to three functional gRNAs can be produced from a single RNA pol III transcript when separated by Csy4 cleavage sites and in the presence of Csy4 in human cells, 2) multiple Csy4-processed gRNAs can be used to direct Cas9 nuclease to introduce multiplex changes in a single human cell, and 3) a functional gRNA flanked by Csy4 cleavage sites can be excised by Csy4 nuclease from a longer mRNA transcript made from an RNA polymerase II promoter.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-20. (canceled)

21. A method of altering expression of a plurality of target genes in a cell, the method comprising expressing a DNA molecule comprising a plurality of sequences encoding guide RNAs (gRNAs), wherein each gRNA comprises a sequence that is complementary to at least 17-20 nts of a target gene, and each gRNA is flanked by at least one Csy4 cleavage sequence comprising or consisting of the sequence GTTCACTGCCGTATAGGCAG (SEQ ID NO: 1).

22. The method of claim 21, wherein the gRNA comprises the sequence:
(SEQ ID NO: 4)
(X₁₇₋₂₀)GUUUUAGAGCUAUGCUGUUUUG(X_{N});
(SEQ ID NO: 5)
(X₁₇₋₂₀)GUUUUAGAGCUA;
(SEQ ID NO: 6)
(X₁₇₋₂₀)GUUUUAGAGCUAUGCUGUUUUG;
(SEQ ID NO: 7)
(X₁₇₋₂₀)GUUUUAGAGCUAUGCU;
(SEQ ID NO: 8)
(X₁₇₋₂₀)GUUUUAGAGCUAGAAAUAGCAAGUUAAAAUAAGGCUAGUCCG
(X_{N});
(SEQ ID NO: 9)
(X₁₇₋₂₀)GUUUUAGAGCUAUGCUGAAAAGCAUAGCAAGUUAAAAUAAGGC
UAGUCCGUUAUC(X_{N});
(SEQ ID NO: 10)
(X₁₇₋₂₀)GUUUUAGAGCUAUGCUGUUUUGGAAACAAAACAGCAUAGCAAG
UUAAAAUAAGGCUAGUCCGUUAUC(X_{N});
(SEQ ID NO: 11)
(X₁₇₋₂₀)GUUUUAGAGCUAGAAAUAGCAAGUUAAAAUAAGGCUAGUCCGU
UAUCAACUUGAAAAAGUGGCACCGAGUCGGUGC(X_{N}),
(SEQ ID NO: 12)
(X₁₇₋₂₀)GUUUAAGAGCUAGAAAUAGCAAGUUUAAAUAAGGCUAGUCCGU
UAUCAACUUGAAAAAGUGGCACCGAGUCGGUGC;
(SEQ ID NO: 13)
(X₁₇₋₂₀)GUUUUAGAGCUAUGCUGGAAACAGCAUAGCAAGUUUAAAUAAG
GCUAGUCCGUUAUCAACUUGAAAAAGUGGCACCGAGUCGGUGC;
or
(SEQ ID NO: 14)
(X₁₇₋₂₀)GUUUAAGAGCUAUGCUGGAAACAGCAUAGCAAGUUUAAAUAAG
GCUAGUCCGUUAUCAACUUGAAAAAGUGGCACCGAGUCGGUGC,
wherein X₁₇₋₂₀ is a sequence complementary to the complementary strand of 17-20 consecutive nucleotides of a target sequence.

23. The method of claim 21, wherein the DNA molecule is operably linked to a promoter sequence.

24. The method of claim 21, wherein the DNA molecule comprises two, three, or more gRNA sequences.

25. The method of claim 23, wherein the promoter sequence is a RNA Polymerase II (Pol II) promoter or Pol III promoter.

26. The method of claim 24, wherein the promoter sequence is a RNA Pol II promoter.

27. The method of claim 26, wherein the Pol II promoter is selected from the group consisting of CAG, EF1A, CAGGS, PGK, UbiC, CMV, B29, Desmin, Endoglin, FLT-1, GFPA, and SYN1 promoters.

28. A method of altering expression of a target gene in a cell, the method comprising expressing a DNA molecule comprising a plurality of sequences encoding guide RNAs (gRNAs), wherein each gRNA comprises a sequence that is complementary to at least 17-20 nts of the target gene, and each gRNA is flanked by at least one Csy4 cleavage sequence comprising or consisting of the sequence GTTCACTGCCGTATAGGCAG (SEQ ID NO: 1).

29. The method of claim 28, wherein the gRNA comprises the sequence:
(SEQ ID NO: 4)
(X₁₇₋₂₀)GUUUUAGAGCUAUGCUGUUUUG(X_{N});
(SEQ ID NO: 5)
(X₁₇₋₂₀)GUUUUAGAGCUA;
(SEQ ID NO: 6)
(X₁₇₋₂₀)GUUUUAGAGCUAUGCUGUUUUG;
(SEQ ID NO: 7)
(X₁₇₋₂₀)GUUUUAGAGCUAUGCU;
(SEQ ID NO: 8)
(X₁₇₋₂₀)GUUUUAGAGCUAGAAAUAGCAAGUUAAAAUAAGGCUAGUCCG
(X_{N});
(SEQ ID NO: 9)
(X₁₇₋₂₀)GUUUUAGAGCUAUGCUGAAAAGCAUAGCAAGUUAAAAUAAGGC
UAGUCCGUUAUC(X_{N});
(SEQ ID NO: 10)
(X₁₇₋₂₀)GUUUUAGAGCUAUGCUGUUUUGGAAACAAAACAGCAUAGCAAG
UUAAAAUAAGGCUAGUCCGUUAUC(X_{N});
(SEQ ID NO: 11)
(X₁₇₋₂₀)GUUUUAGAGCUAGAAAUAGCAAGUUAAAAUAAGGCUAGUCCGU
UAUCAACUUGAAAAAGUGGCACCGAGUCGGUGC(X_{N}),
(SEQ ID NO: 12)
(X₁₇₋₂₀)GUUUAAGAGCUAGAAAUAGCAAGUUUAAAUAAGGCUAGUCCGU
UAUCAACUUGAAAAAGUGGCACCGAGUCGGUGC;
(SEQ ID NO: 13)
(X₁₇₋₂₀)GUUUUAGAGCUAUGCUGGAAACAGCAUAGCAAGUUUAAAUAAG
GCUAGUCCGUUAUCAACUUGAAAAAGUGGCACCGAGUCGGUGC;
or
(SEQ ID NO: 14)
(X₁₇₋₂₀)GUUUAAGAGCUAUGCUGGAAACAGCAUAGCAAGUUUAAAUAAG
GCUAGUCCGUUAUCAACUUGAAAAAGUGGCACCGAGUCGGUGC,
wherein X₁₇₋₂₀ is a sequence complementary to the complementary strand of 17-20 consecutive nucleotides of a target sequence.

30. The method of claim 28, wherein the DNA molecule is operably linked to a promoter sequence.

31. The method of claim 28, wherein the DNA molecule comprises two, three, or more gRNA sequences.

32. The method of claim 30, wherein the promoter sequence is a RNA Polymerase II (Pol II) promoter or Pol III promoter.

33. The method of claim 30, wherein the promoter sequence is a RNA Pol II promoter.

34. The method of claim 33, wherein the Pol II promoter is selected from the group consisting of CAG, EF1A, CAGGS, PGK, UbiC, CMV, B29, Desmin, Endoglin, FLT-1, GFPA, and SYN1 promoters.

35. A method of altering expression of a target gene in a cell by targeting multiple parts of a single gene, the method comprising expressing a DNA molecule comprising a plurality of sequences encoding guide RNAs (gRNAs), wherein each gRNA comprises a sequence that is complementary to at least 17-20 nts of the target gene, and each gRNA is flanked by at least one Csy4 cleavage sequence comprising or consisting of the sequence GTTCACTGCCGTATAGGCAG (SEQ ID NO:1).

36. The method of claim 35, wherein the gRNA comprises the sequence:
(SEQ ID NO: 4)
(X₁₇₋₂₀)GUUUUAGAGCUAUGCUGUUUUG(X_{N});
(SEQ ID NO: 5)
(X₁₇₋₂₀)GUUUUAGAGCUA;
(SEQ ID NO: 6)
(X₁₇₋₂₀)GUUUUAGAGCUAUGCUGUUUUG;
(SEQ ID NO: 7)
(X₁₇₋₂₀)GUUUUAGAGCUAUGCU;
(SEQ ID NO: 8)
(X₁₇₋₂₀)GUUUUAGAGCUAGAAAUAGCAAGUUAAAAUAAGGCUAGUCCG
(X_{N});
(SEQ ID NO: 9)
(X₁₇₋₂₀)GUUUUAGAGCUAUGCUGAAAAGCAUAGCAAGUUAAAAUAAGGC
UAGUCCGUUAUC(X_{N});
(SEQ ID NO: 10)
(X₁₇₋₂₀)GUUUUAGAGCUAUGCUGUUUUGGAAACAAAACAGCAUAGCAAG
UUAAAAUAAGGCUAGUCCGUUAUC(X_{N});
(SEQ ID NO: 11)
(X₁₇₋₂₀)GUUUUAGAGCUAGAAAUAGCAAGUUAAAAUAAGGCUAGUCCGU
UAUCAACUUGAAAAAGUGGCACCGAGUCGGUGC(X_{N}),
(SEQ ID NO: 12)
(X₁₇₋₂₀)GUUUAAGAGCUAGAAAUAGCAAGUUUAAAUAAGGCUAGUCCGU
UAUCAACUUGAAAAAGUGGCACCGAGUCGGUGC;
(SEQ ID NO: 13)
(X₁₇₋₂₀)GUUUUAGAGCUAUGCUGGAAACAGCAUAGCAAGUUUAAAUAAG
GCUAGUCCGUUAUCAACUUGAAAAAGUGGCACCGAGUCGGUGC;
or
(SEQ ID NO: 14)
(X₁₇₋₂₀)GUUUAAGAGCUAUGCUGGAAACAGCAUAGCAAGUUUAAAUAAG
GCUAGUCCGUUAUCAACUUGAAAAAGUGGCACCGAGUCGGUGC,
wherein X₁₇₋₂₀ is a sequence complementary to the complementary strand of 17-20 consecutive nucleotides of a target sequence.

37. The method of claim 35, wherein the DNA molecule is operably linked to a promoter sequence.

38. The method of claim 35, wherein the DNA molecule comprises two, three, or more gRNA sequences.

39. The method of claim 37, wherein the promoter sequence is a RNA Polymerase II (Pol II) promoter or Pol III promoter.

40. The method of claim 39, wherein the promoter sequence is a RNA Pol II promoter, wherein the RNA Pol II promoter optionally is selected from the group consisting of CAG, EF1A, CAGGS, PGK, UbiC, CMV, B29, Desmin, Endoglin, FLT-1, GFPA, and SYN1 promoters.
